(19) United States
(12) Patent Application Publication
(10) Pub. No.: US 2002/0156129 A1
(43) Pub. Date: Oct. 24, 2002

(54) Method of treating pruritus and a pharmaceutical composition for the method

(76) Inventors: Yasushi Kuraishi, Sugitani (JP); Takayuki Miyamoto, Nakaniikawa-gun (JP)

Correspondence Address: CHRISTIE, PARKER & HALE, LLP, P.O. BOX 7068, PASADENA, CA, 91109-7068, US

(21) Appl. No.: 09/982,380

(22) Filed: Oct. 17, 2001

(30) Foreign Application Priority Data
Feb. 21, 2001 (JP) 2001-44656

(51) Int. Cl. A61K 31/198; A61K 31/21; A61K 31/22

(52) U.S. Cl. 514/551; 514/565; 514/509

(57) ABSTRACT

The object of the present invention is to provide a pharmaceutical composition for treatment of pruritus not associated with dermal inflammation such as pruritus caused by the decrease of cutaneous barrier function. Particularly, the object of the present invention is to provide a pharmaceutical composition, comprising at least one substances having the inhibitory function of NO activity in vivo, such as the inhibitory activity for NO biosynthesis and/or at least one substances having the eliminating activity for NO and a pharmaceutically acceptable carrier.

Another object of the present invention is to provide a method of treatment of pruritus not associated with dermal inflammation such as pruritus caused by the decrease of skin barrier function. The method comprises the step of administrating the pharmaceutical composition of the present invention.

### CROSS-REFERENCE TO RELATED APPLICATION(S)

This Patent Application claims priority of Japanese Patent Application: No. 2001-044656, filed on Feb. 21, 2001.

### BACKGROUND OF THE INVENTION

The present invention relates to a pharmaceutical composition for treating pruritus in dermatosis characterized by hypo-function of cutaneous barrier such as senile melanoderma, or pruritus caused by xeroderma from hepatopathy and the like. The present invention also relates to a method for treating pruritus in dermatosis characterized by hypo-function of cutaneous barrier such as senile melanoderma, or pruritus caused by xeroderma from hepatopathy and the like.

More particularly, the present invention relates to a pharmaceutical composition for treating pruritus which cannot be treated effectively by conventional anti-pruritic agents such as histamine H1 receptor antagonists.

The term Dry skin is the generic for expressing the condition of a skin having a reduced hydration of the stratum corneum caused by increase of transepidermal water loss which leads to the cutaneous barrier disruption. Causes of the dry skin are aging of the skin such as senile melanoderma (Journal of Clinical Investigation, Vol. 95 (5), 2281-2290, 1995), and endogenous factors such as chronic renal failure (Nephrology, Dialysis, Transplantation, Vol. 9 (9), 1302-1304, 1994) and cholestatic hepatopathy. In addition, it is known that the dry skin of a healthy person is also caused by exogenous factors such as abnormally dry external environment in winter, and physical and scientific factors such as ungreasing of the sebaceous envelope caused by an excessively high frequent bathing and use of a solvent or a surfactant. One of the common symptoms of the patients with such hypo-function of cutaneous barrier is a systemic or local itching. This condition arises problems of serious unpleasant feeling and increment of the skin manifestations by the disruption of the barrier function caused by scratching.

On the other hand, nitric oxide (NO) found in organisms is a very unstable free radical molecule and the half-life thereof is supposed to be a few seconds. The biosynthesis of nitrogen monoxide is catalyzed by nitric oxide synthase (NOS) and is produced from essential amino acid L-arginine as a substrate. Therefore, use of several agents is known as approaches for inhibiting the physiological effects of NO. Such approaches include the inhibition by the use of L-arginine analog including L-type derivatives of L-arginine wherein the guanidino group of L-arginine is linked to methyl, amino, nitro and other functional groups such as Nw-nitro-L-arginie methyl ester (L-NAME), or by the use of specific inhibitors of NOS activity such as S-methylisothiourea and the use of agents being capable of binding to the free radical and eliminate it, such as carboxyl-PTIO or hemoglobin.

NO is originally found as a gas mediator having a smooth muscle relaxant activity, and is revealed to function as an apoptosis inducer released from inflammatory leukocytes such as monocytes during an inflammation or to function an signal transducer in vivo. It is also known that there are two types of NOS, namely, constitutive NOS and inducible NOS. It is also observed in the skin that NOS can be induced at exanthesis sites in inflammatory dermatosis such as atopic dermatitis or at the cutaneous sites damaged by ultraviolet ray radiation. Furthermore, NO is also found to be exist in the brain nerve systems and is revealed to function as a neurotransmitter.

Thus, it is presumed that inhibiting the function of NO in vivo may have therapeutic effects on different inflammatory diseases. This idea is similarly applicable to inflammatory dermatosis. For example, it is reported that NOS expression was found in the epidermis of exanthesis sites for NC mouse which is the model mouse exhibiting the skin lesion resembled atopic dermatitis, and the scratching action can be suppressed by administrating L-NAME, an inhibitor of NOS (Journal of Japan Pharmacology Society, 114 (suppl. 1), 17-21, 1999). It is also clinically reported that L-NA, which is the inhibitor of NOS was experimentally used in a local drug treatment for the purpose of treating pruritus in the patients suffering from atopic dermatitis (International Journal of Dermatology, Vol. 34 (4), 292-295, 1995). However, it is still unknown whether the therapeutic effect is the result of the suppression of inflammatory pathological changes of skin caused by NO or is the pruritus-specific suppressive effects resulted from the decrease of signal transducing activity cause by NO. Therefore, these drugs have not been positively used for pruritus caused by other causes.

The mechanism of onset of pruritus has previously been believed to be that an irritating substance and an allergen easily invades the skin when the barrier function weakens and arises the degranulation of cutaneous mast cells, which leads to the onset of pruritius. However, in the case of pruritus associated with dermatosis caused by the hypo-function of cutanesou barrier, cutaneous reactions such as erythema or wheal caused by the degranulation of cutaneous mast cells, are not observed. Thus, the detailed onset mechanism has not yet been revealed. Actually, the pruritus caused by said dermatosis cannot be blocked by a histamine H1 receptor antagonist generally used for relieving the pruritus caused by the degranulation of intradermal mastocytes in many cases. Although it was reported that, for example, an opioid receptor antagonist used in a trial administration was effective (Annals of Internal Medicine, Vol. 123 (3), pages 161 to 167, 1995), effective method of treatment has not yet been established.

### SUMMARY OF THE INVENTION

The inventors have developed the experimental procedures to produce non-inflammatory pruritus on small experimental animals resulted from cutaneous symptom similar to above described dermatosis and investigated the substances and methods of treatment for pruritus which may be effective on the model animals produced by the procedures.

Therefore the object of the present invention is to provide a pharmaceutical composition for treatment for pruritus not associated with dermal inflammation (non-inflammatory pruritus) such as pruritus caused by the decrease of cutaneous barrier function.

Another object of the present invention is to provide a method of treating non-inflammatory puritus such as pruritus caused by the decrease of skin barrier function.

More particularly, the object of the present invention is to provide a pharmaceutical composition, comprising at least one substances having the inhibitory function of NO activity in vivo, such as the inhibitory activity for NO biosynthesis and/or at least one substances having the eliminating activity for NO and a pharmaceutically acceptable carrier.

Especially, the pharmaceutical composition of the present invention comprises at least one of the substances having the activity selected from the group consisting of:

A. an inhibitory activity for nitric oxide biosynthesis in vivo, or

B. an eliminating activity for nitric oxide due to the binding to nitrogen monoxide; and

C. a pharmaceutically acceptable carrier.

The further object of the present invention is to provide a method for treating oninflammatory pruritus, which comprises administrating at least one substances having the inhibitory activity of NO function in vivo, such as the inhibitory activity for NO biosynthesis and/or at least one substances having the eliminating activity for NO.

Especially, the method of the present invention comprises administrating at least one of the substances having the activity selected from the group consisting of:

A. an inhibitory activity for nitric oxide biosynthesis in vivo, or

B. an eliminating activity for nitric oxide due to the binding to nitrogen monoxide to a patient suffering from the pruritus.

### BRIEF DESCRIPTION OF THE DRAWINGS

FIG. 1: Shows the number of scratching of mice treated with acetone/diethylether mixture and distilled water twice daily for 5 days, followed by subcutaneous administration of L-NAME or D-NAME at the dose of 1 mg/kg for 15 minutes before recording the scratching action. A number of scratching action of 8 mice per 2 hours is shown by the average percentage±standard error based on the average of the control group (100%). “S” represents the control group which received saline, “L” represents L-NAME administrated group and “D” represents D-NAME administrated group. Symbol “*” in FIG. 1 indicates that the difference from the control group is statistically significant (p<0.05).

FIG. 2: Shows the number of scratching of mice treated with acetone/diethylether mixture and distilled water twice daily for 5 days, followed by topical application of 50 ml of 5% solution of L-NAME or D-NAME 1 hour before recording the scratching action. A number of scratching action of 8 mice per 2 hours is shown by the average percentage±standard error based on the average of the control group (100%). Symbol “V” represents the control group which received saline, “L” represents L-NAME applied group and “D” represents D-NAME applied group. Symbol “*” in FIG. 2 indicates that the difference from the control group is statistically significant (p<0.05).

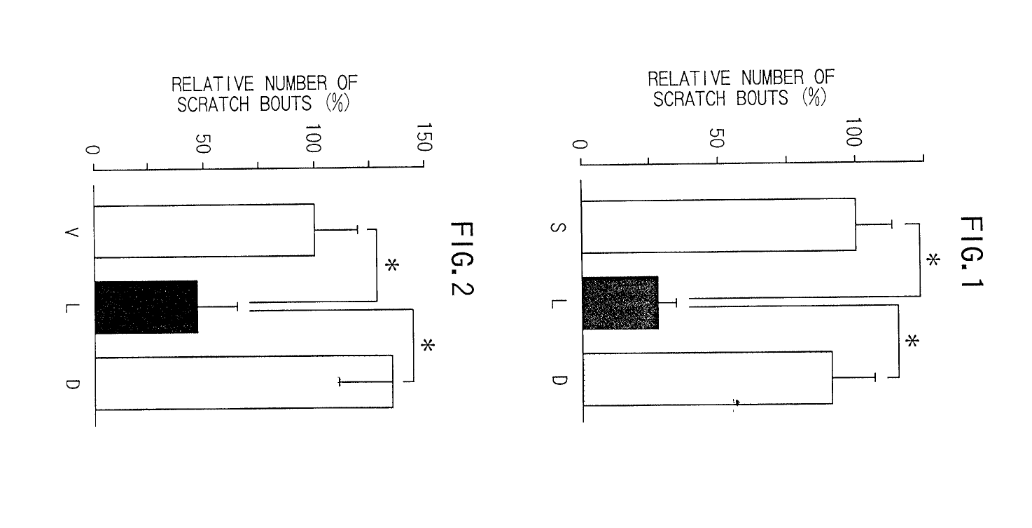

### DETAILED DESCRIPTION OF THE INVENTION

The active ingredients which may be contained in the pharmaceutical composition of the present invention include amino acid L-arginine analogs, which are the substrates and the materials for NO synthesis, such as L-derivatives of L-arginine wherein the guanidino group of L-arginine is linked to methyl, amino, nitro and other functional groups such as Nw-nitro-L-arginine methyl ester (L-NAME), Nw-monomethyl-L-arginine(L-NMMA), Nw-nitro-arginine, Nw-allyl-L-arginine, Nw-cyclopropyl-L-arginine, Nw-amino-L-arginine, Nw-nitro-L-arginine-p-nitroanilide and Nw, Nw-dimethylarginine, Although these substances do not inhibit the catalytic activity of NOS, they can inhibit biosynthesis of NO because NO cannot be produced from these substances.

Alternately, the active ingredients which may be also contained in the pharmaceutical composition of the present invention include substances which have the activity of NO synthase inhibitors, such as 2-iminobiotin, L-thiocitruline, L-homothiocitruline, S-methyl-L-thiocitruline, S-ethyl-L-thiocitruline, S-methylisothiourea, S-ethylisochiourea, S-isopropylisothiourea, S,S (1,3-phenilene bis (1,2-ethanediyl)) bis isothiourea, 2-amino thiazoline, 2-aminothiazole, N-(3-(aminomethyl)benzyl)-acetamidine, N (-(4,5-dihydrothiazole-2-yl) ornithine, N (-iminoethyl-L-ornithine, L-N6-(1-iminoehtyl) lysine, AR-R17477, HMN-1180, (2-trifluoromethylphenyl) imidazole, 7-nitroindazole, 6-nitroindazole or indazole. These substances can also inhibit NO synthesis catalyzed by NOS.

The pharmaceutical composition of the present invention may also contain substances which can eliminate NO by binding to NO, such as carboxy-2-phenyl-4,4,5,5-tetramethyl-imidazoline-1-oxyl-3-oxide (carboxyl-PTIO) or hemoglobin. These substances can eliminate the physiological function of NO which was biosynthesized, and can thereby develop the effectiveness in the treatment of pruritus.

The pharmaceutical composition of the present invention may comprise one or more of these substances When the pharmaceutical composition of the present invention comprises more than one substances, the substances may have the same or different mechanism of action and/or they may have the same or different point of action.

The pharmaceutical composition of the present invention may be administrated by injection, systemic administration such as oral administration or local administration such as local hypodermic injection or external use. Preferably, the pharmaceutical composition of the present invention may be administrated locally to the pruritus sites. The antipruritic formulation for external use may contain 0.001%-30% by weight, preferably 0.01-20% by weight of above described substances as active ingredients.

The peroral agent for treating pruritus according to the present invention may be in any formulation used as peroral formulation. For example, the pharmaceutical composition of the present invention may be in the form of tablet, powder, granule, syrup, capsule and the like. The pharmaceutical composition may further include conventional excipients depending on the formulation and may include appropriate auxiliaries. The external agent for treating pruritus according to the present invention may be in any formulation used as formulation for external use. For example, the pharmaceutical composition may be in the form of ointment, cream, gel, lotion, liquid, patch, cataplasm, aerosol, linimentum and the like. The pharmaceutical composition of the present invention may further include conventional excipients depending on the formulation and may include appropriate auxiliaries, such as suspending agents including arabian gum, sodium arginate, carboxymethyl cellulose sodium, methyl cellulose or bentonite; emulsifiers including sodium lauryl sulfate, polysorbates, sorbitan mono-fatty acid esters, polyoxyethylene fatty acid esters and the like.

For the ointments of the present invention, any of oleaginous base, water-soluble base, emulsion base or gel base may be used. The oleaginous base may be mineral base including yellow petrolatum, white petrolatum, paraffin, liquid paraffin, platinate base, silicone and the like or bases of animals and plants origin including plant oil, lard, tallow, wax and the like. The emulsion base may be a cream comprising various oils, surfactants and water. The water soluble base may comprise polyethylene glycol as a main base.

The pharmaceutical composition for external use according to the present invention may also comprise benzyl alcohol, crotamiton, polyethylene glycol fatty acid esters, glycols such as propylene glycol, butylene glycol, polyethylene glycol and the like).

Since the role of NO is believed not to be the direct cause of pruritus but is supposed to be an enhancer for pruritus, the pharmaceutical composition for treating pruritus of the present invention may be administrated or formulated with other antipruritic ingredients. The other antipruritic ingredients may be histamine H1 receptor antagonists such as diphenhydramine or chlorpheniramine, local anesthetics such as procaine, lidocaine, dibucaine, non-specific antipruritic agents such as crotamiton or anti-inflammatory agents such as adrenal cortical hormones, acetylsalicylic acid, indomethacin, diclofenac, bufexamac, ibuprofenpiconol.

The pharmaceutical composition for treating pruritus according to the present invention may also be formulated with urea, glycerin, sodium lactate, sodium pyrrolidone carboxylate, amino acids, heparinoids, g-oryzanol, ceramides or squalenes as a moisturizing agent for complementing the barrier function of stratum corneum.

The pharmaceutical composition for treating pruritus according to the present invention may be applied to pruritus caused mainly by the dehydration of skin due to the hypofunction of cutaneous barrier, preferably to puritus associated with dehydration of skin, such as senile prutitus or pruritus associated with cholestatic hepatopathy and the like. For treating dermatosis accompanied by inflammatory exanthesis, the pharmaceutical composition for treating pruritus according to the present invention is preferably used together with other antipruritus agents or is administrated in the formulation containing other antipruritus agents.

The evaluation of the therapeutic effects of the pharmaceutical composition of the present invention may be conducted by using the small test animals produced by the method developed by the inventors, such as mice of which the cutaneous barrier function have been disrupted. Such animals may be produced according to the following procedure: An noninflammation dermal lesion caused by the disruption of cutaneous barrier function may be induced by degreasing the small test animals by applying an organic solvent to the glabrous skin of the animals and then treating with water. The organic solvent used for the treatment is not particularly limited so far as the solvent per se does not have a strong corroding effect on the skin. The organic solvents include alcohols, ketones, ethers and esters, preferably aliphatic hydrocarbons and DMSO, more preferably, acetone or mixtures of acetone and ether, and particularly preferably a mixture of acetone and diethylether in a ratio of 1:1. In the treatment with the organic solvent or water, the part to be treated is covered with an absorbent using a cotton containing it, and then the superfluous solvent or water is wiped off or dripped off. Preferably, the part is covered with the absorbent cotton containing the solvent or water and then the superfluous solvent or water is wiped off. The time required for the treatment is not strictly limited and may vary depending on the kind of the small animals and the organic solvent used. The time required for the treatment with the organic solvent is generally at least several seconds, preferably about several seconds to several minutes, and more preferably about 10 to 60 seconds. The time required of the treatment with water is at least 30 seconds, preferably about 30 seconds to several minutes, and particularly about 30 to 60 seconds. The treatment is repeated until the stratum corneum has become whity and powder-coated or, in other words, until the formation of wrinkles or scales is observed on the skin. The intervals between the treatment with the organic solvent and the subsequent treatment with water are shorter than a period in which the barrier function is completely recovered. The treatments are desirably continuously repeated with such a frequency that the inflammatory stimulation is not given to the skin until at least skin manifestations caused by drying such as scale have become to be observed. The frequency of the treatments varies depending on the kind of the small animals. Usually, the treatments are repeated sequentially with a frequency of 1 to 3 times a day for several days (preferably at least for 3 days). The onset of the dermatosis caused by the disruption of the barrier function can be confirmed by the observation of the appearance of the skin, and the determination of hydration of the stratum corneum and transepidermal water loss according to the present invention. A combination of two or more methods is desirable. For example, the hydration of the stratum corneum can be determined by a technique of conductimetry, determination of electrostatic capacity or FT-IR method. Transepidermal water loss can be non-invasively determined with an electric measuring instrument or the like so as not to influence the scratching action. The transepidermal water loss is desirably still increasing one day after the barrier disrupting treatment, unlike the untreated animals. Preferably, the transepidermal water loss of the treated animals is at least twice as much as that of the untreated animals. On the other hand, hydration of the stratum corneum of the treated animals is desirably lower than that of the untreated animals one day after the barrier disrupting treatment. Namely, hydration of the treated animals determined on the basis of the electrostatic capacity is preferably not more than ½ of that of the untreated animals one day after the barrier disrupting treatment.

The therapeutic effects of the composition of the present invention can be evaluated on the basis of the changes in the condition of skin or the decrease in the number of scratching actions, after applying the pharmaceutical composition of the present invention in the form of the above described formulations and doses to such obtained small test animals. Although the number of scratching action can be determined by direct visual observation, it is preferably observed and recorded in an unattended environment. For example, the observation and recording are desirably carried out by recording the action of the animals in a cage having an open or transparent top with a video camera placed above the cage. Particularly, one scratching action means a series of action beginning when the small animal raises its hind limb for starting the scratching and ending when the animal lowers its hind limb. The scratching action is determined by counting the number of scratching the region where the barrier function is disrupted or the surrounding region with the hind limbs. The determination period for each animal is at least 30 minutes, preferably 30 to 150 minutes. Several small animals, usually about 4 to 12 animals, are used for each test and the results are statistically processed.

Animals which did not receive the medicine may be used as a control group. Animals which received a histamine H1 receptor antagonist which is used for suppressing the pruritus caused by the degranulation of intracutaneous mast cells, can be used as a comparative group.

Determining the number of the scratching action by such methods, the inhibitory effects of test compositions may be confirmed if the scratching action statistically significantly reduced (significant level 5%). Also, in the diagnosis of the skin appearance, the suppression of the formation of scales or the like or appearance thereof may be employed as indices. When no abnormal finding is obtained at all, the composition may be determined to have a remarkable effect. Furthermore, as for hydration or the stratum corneum, the effect of test compositions may be determined based on the protection or recovery from lowering of hydration caused by the barrier-disrupting process, and the tested composition may be determined to have a remarkable effects when the water the hydration after administration is substantially equal to that determined before the cutaneous barrier disrupting treatment. Preferably, the effects of the tested compositions are evaluated based on the combination of such measurement results and findings.

### EXAMPLES

The following examples illustrate the inhibitory effects of the pharmaceutical compositions of the present invention on pruritus model of small test animals. These examples are provided to illustrate the claimed invention and they are not intended to limit the spirit and the scope of the present invention.

### Example 1

A rostral part (4 cm2, i.e. 2×2 cm) of the back skin in each of ICR mice was shaved. Four days after the shaving, the following test was started: The skin was covered with a 2 cm×2 cm absorbent cotton impregnated with a mixture of acetone and diethylether (1:1) for 15 seconds. The skin was wiped to remove superfluous solvent, then covered with an absorbent cotton impregnated with distilled water for 30 seconds, and wiped in the same manner as that described above. This treatment for disrupting the barrier function of the skin was conducted twice in daily at intervals of at least 8 hours for 5 days. On the next day after the completion of the 5 days treatment for disrupting the cutaneous barrier function, each mouse was placed in each section of an acrylic acid resin cage (26×18×33 cm) divided into four sections (each section: 13×9×33 cm). After the mice was acclimatized to be in the cage in unattended environment for 45 minutes, the test substance Nw-nitro-L-arginine methyl ester (L-NAME) or the control substance Nw-nitro-D-arginine methyl ester (D-NAME) in saline or saline alone was subcutaneously administered (10 mg/kg) at the location where the barrier-disrupting treatment was applied. After being acclimatized for 15 minutes more, the action of each mouse was filmed and recorded with a 8 mm video camera placed above the cage in such a manner that the 4 sections are in one scene or 2 hours. The scratching action was observed by playing back the video tape. The number of the scratching action by which the tip of a toe of a hind limb of the mouse touched the shaved part of the skin in 2 hours was counted by the visual observation. One scratching action was a series of action beginning when the mouse raised its hind limb for starting the scratching and ending when the animal lowers its hind limb. The inhibitory effect of the pharmaceutical compositions was evaluated by using the relative percentage of the numbers of scratching action as an index, based on the mean numbers of scratching actions of the control group represented as 100%.

The results of the test were as follows:

The number of the scratching action in the group to which L-NAME was administrated, was reduced to 27.9±6.4% of the control group to which saline was administrated. The difference between the two groups was statistically significant. On the other hand, the number of the scratching action was 90.9±15.2% in the group to which D-NAME was administered, which was not statistically significant. The difference between L-NAME administrated group and D-NAME administrated group was statistically significant.

Therefore, it was suggested that the inhibition of L-NAME is the result of the inhibition of NO synthesis, because the scratching action was only inhibited by L-NAME and was not inhibited by the subcutaneous administration of equivalent amount of D-NAME.

### Example 2

On the next day after the completion of the 5 day barrier function disrupting treatment on the test animals such as those used in Example 1, the scratching action of mice was recorded for 120 minutes in the same manner as described in Example 1. The observation of the behavior of the animals and the measurement of the number of the scratching action were conducted in the same manner as described in Example 1. 60 minutes before recording the scratching action, that is, before the animals were acclimatized to the environment, 50 ml of 50% ethanol solution of the test substances, Nw-nitro-arginine methyl ester(L-NAME) or the control substances, Nw-nitro-D-arginine methyl ester (D-NAME), or 50 ml of the solvent (50% ethanol) alone was applied respectively at the portion where the barrier-disrupting treatment was applied. The inhibitory effect of the substances on the scratching action was indicated by the relative number of relative scratching based on the number of scratching action in the control group received only the solvent as 100%.

The test results were as follows:

The number of the scratching action in the L-NAME applied group was reduced to 47.4±17.8% based on that of the control group. The difference among groups was statistically significant. On the other hand, the number of the scratching action in the L-NAME applied group was 134±24.7% compared to the control group, which means that there was no significant difference between these groups. The difference between L-NAME applied groups and D-NAME applied groups was statistically significant.

Therefore, the inhibitory effect of L-NAME application on scratching action is considered to be the result of the inhibition of NO synthesis, because the scratching action was inhibited by L-NAME, while it was not inhibited by applying equivalent amount of D-NAME. Also the site of the action of L-NAME functions is considered to be local cutaneous regions, because the application on the sites exhibiting dermal symptom was effective. Therefore, it was understood from these results that the inhibition of NO synthesis at local cutaneous regions is effective for the inhibition of pruritus.

### Example 3

Nw-nitro-L-arginine methyl ester was mixed and agitated at 5% by weight with the base solution for the liquid type composition for external application containing the ingredients shown in Table 1 to make the liquid type pharmaceutical composition for external application.

TABLE 1

| Base solution for liquid type | |
|---|---|
| composition for external application | Percentage by weight |
| Ethyl alcohol | 50% |
| Purified water | 50% |
| Total | 100% |

### Example 4

Diphenhydramine hydrochloride and S—methylisothiourea were mixed and agitated at 1.0% by weight respectively with the base for the ointment containing the ingredients shown in Table 2 to make the ointment conatining Diphenhydramine hydrochloride and S-methylisothiourea at 1.0% by weight respectively.

TABLE 2

| Base for ointment | Percentage by weight |
|---|---|
| White petrolatum | 25.0% |
| Steariyl alcohol | 20.0% |
| Propylene glycol | 12.0% |
| Polyoxyethylene hydrogenated castor | 4.0% |
| oil | |
| Monostearate glycerol | 1.0% |
| Methyl parahydroxybenzoate | 0.1% |
| Propyl parahydroxybenzoate | 0.1% |
| Purified water | |
| Total | 100% |

### Example 5

Carboxy—PTIO and urea were mixed and agitated at 2.0% and 10.0% by weight, respectively, with the base for the ointment containing the ingredients shown in Table 3 to make the ointment containing carboxy-PTIO and urea were mixed and agitated at 2.0% and 10.0% by weight, respectively.

TABLE 3

| Base for ointment | Percentage by weight |
|---|---|
| Carboxyvinyl polymer | 1.0% |
| Benzylalcohol | 0.5% |
| Octyldodecanol | 5.0% |
| Fatty acid ester of polyethylene | 0.5% |
| glycol | |
| Diisopropanolamine | 0.7% |
| Edetic acid | 0.01% |
| Purified water | |
| TOTAL | 100% |

The present invention provides a pharmaceutical composition effective to pruritus which is associated with dry skin caused by the disruption of cutaneous barrier function and which is not associated with allergic inflammation. The present invention also provides a method for treating pruritus which is associated with dry skin caused by the disruption of cutaneous barrier function and which is not associated with allergic inflammation. Particularly, the present invention provides a pharmaceutical composition and a therapeutic method for effective to pruritus to which conventional histamine H1 antagonist is ineffective, and thereby it becomes possible to treat pruritus such as pruritus in xeroderma or chronic renal failure.

Consequently, the present invention may contribute to the improvement of quality of life or the relief of pains of patients.

It is also understood that the examples and embodiments described herein are only for illustrative purpose, and that various modifications will be suggested to those skilled in the art without departing from the spirit and the scope of the invention as hereinafter claimed.

What is claimed is:

1. A pharmaceutical composition comprising one or more substances having an activity of inhibiting an effect of nitric oxide in vivo and a pharmaceutically acceptable carrier.

2. The pharmaceutical composition of claim 1, wherein the substance having an activity of inhibiting the effect of nitric oxide in vivo is a substances having the activity of inhibiting the biosynthesis of nitric oxide in vivo.

3. The pharmaceutical composition of claim 2, wherein the substance having the activity of inhibiting the biosynthesis of nitric oxide in vivo is a substrate analog of nitrogen monoxide synthase.

4. The pharmaceutical composition of claim 2, wherein the substance having the activity of inhibiting the biosynthesis of nitric oxide in vivo is a substance having the activity of inhibiting the catalytic activity of nitric oxide synthase.

5. The pharmaceutical composition of claim 1, wherein the substance having an activity of inhibiting the effect of nitric oxide in vivo is a substances having the activity of eliminating nitric oxide in vivo.

6. The pharmaceutical composition of claim 3, wherein the substrate analog of nitric oxide synthase is selected from the group consisting of Nw-nitro-L-arginine methyl ester (L-NAME), Nw-monomethyl-L-arginine (L-NMMA), Nw-itro-arginine, Nw-allyl-L-arginine, Nw-cyclopropyl-L-arginine, Nw-amino-L-arginine, Nw-nitro-L-arginine-p-nitroanilide and Nw, Nw-dimethylarginine.

7. The pharmaceutical composition of claim 4, wherein the substance having the activity of inhibiting the catalytic activity of nitric oxide synthase is selected from the group consisting of 2-iminobiotin, L-thiocitruline, L-homothiocitruline, S-methyl-L-thiocitruline, S-ethyl-L-thiocitruline, S-methylisothiourea, S-ethylisochiourea, S-isopropylisothiourea, S,S (1,3-phenilenebis (1,2-ethanediyl)) bisisothiourea, 2-amino thiazoline, 2-aminothiazole,-(3-(aminomethyl)benzyl)-acetamidine, N (-(4,5-dihidrothiazole-2-yl) ornithine, N (-iminoethyl-L-ornithine, L-N6-(1-iminoehtyl)-lysine, AR-R17477, HMN-1180, (2-trifluoromethylphenyl) imidazole, 7-itroindazole, 6-nitroindazole and indazole.

8. The pharmaceutical composition of claim 5, wherein the substance having the activity of eliminating nitric oxide in vivo is selected from the group consisting of carboxy-2-phenyl-4,4,5,5-tetramethyl-imidazoline-1-oxyl-3-oxide and hemoglobin.

9. The pharmaceutical composition of claim 1 which further comprises one or more agents selected from the group consisting of a histamine H1 receptor antagonist, a local anesthetic and an anti-inflammatory agent.

10. A method of treating noninflammatory pruritus, comprising the step of administrating one or more substances having an activity of inhibiting an effect of nitric oxide in vivo to a patient suffering from the pruritus.

11. The method of claim 10, wherein the substance having an activity of inhibiting the effect of nitric oxide in vivo is a substance having the activity of inhibiting the biosynthesis of nitric oxide in vivo.

12. The method of claim 11, wherein the substance having the activity of inhibiting the biosynthesis of nitric oxide in vivo is a substrate analog of nitric oxide synthase.

13. The method of claim 11, wherein the substance having the activity of inhibiting the biosynthesis of nitric oxide in vivo is a substance having the activity of inhibiting the catalytic activity of nitric oxide synthase.

14. The method of claim 10, wherein the substance having an activity of inhibiting the effect of nitric oxide in vivo is a substance having the activity of eliminating nitric oxide in vivo.

15. The method of claim 12, wherein the substrate analog of nitric oxide synthase is selected from the group consisting of Nw-nitro-L-arginine methyl ester (L-NAME), Nw-monomethyl-L-arginine (L-NMMA), Nw-itro-arginine, Nw-allyl-L-arginine, Nw-cyclopropyl-L-arginine, Nw-amino-L-arginine, Nw-nitro-L-arginine-p-nitroanilide and Nw, Nw-dimethylarginine.

16. The method of claim 13, wherein the substance having the activity of inhibiting the catalytic activity of nitric oxide synthase is selected from the group consisting of 2-iminobiotin, L-thiocitruline, L-homothiocitruline, S-methyl-L-thiocitruline, S-ethyl-L-thiocitruline, S-methylisothiourea, S-ethylisochiourea, S-isopropylisothiourea, S,S (1,3-phenilenebis (1,2-ethanediyl)) bis-isothiourea, 2-amino thiazoline, 2-aminothiazole, N-(3-(aminomethyl)benzyl)-acetamidine, N(-(4, 5-dihidrothiazole-2-yl)-ornithine, N(-iminoethyl-L-ornithine, L-N6-(1-iminoehtyl)-lysine, AR-R17477,HMN-1180, (2-trifluoromethylphenyl)-imidazole, 7-nitroindazole, 6-nitroindazole and indazole.

17. The method of claim 14, wherein the substance having the activity of eliminating nitric oxide in vivo is selected from the group consisting of carboxy-2-phenyl-4,4,5,5-tetramethyl-imidazoline-1-oxyl-3-oxide and hemoglobin.

18. A method of treating noninflammatory and inflammatory pruritus, comprising the step of administrating one or more substances having an activity of inhibiting an effect of nitric oxide in vivo and one or more agents selected from the group consisting of a histamine Hl receptor antagonist, a local anesthetic and an anti-inflammatory agent to a patient suffering from the pruritus.
